(19) United States
(12) Patent Application Publication
(10) Pub. No.: US 2009/0191644 A1
(43) Pub. Date: Jul. 30, 2009

(54) Imprinted polymer for binding of organic molecules or metal ions

(76) Inventor: Miruna Petcu, Hamilton (NZ)

Correspondence Address: JACOBSON HOLMAN PLLC, 400 SEVENTH STREET N.W., SUITE 600, WASHINGTON, DC, 20004, US

(21) Appl. No.: 11/631,105

(22) PCT Filed: Jun. 29, 2005
(86) PCT No.: PCT/NZ2005/000145

§ 371 Date: Apr. 4, 2008

(30) Foreign Application Priority Data
Jun. 29, 2004 (NZ) 533818

(51) Int. Cl. G01N 33/566; C08L 39/04; C08L 89/00; G01N 33/20; G01N 33/44

(52) U.S. Cl. 436/501; 525/326.8; 525/54.1; 436/73; 436/85; 436/80

(57) ABSTRACT

The invention relates to an imprinted polymer imprinted with an organic molecule or a metal ion wherein the matrix of the polymer has been prepared from one or more monomers including bilirubin or an analogue thereof. The imprinted polymers may be prepared by polymerising one or more monomers including bilirubin or an analogue or derivative thereof in the presence of the molecule or metal ion to be imprinted or an analogue or derivative thereof, and subsequently at least partly removing the molecule or ion to be imprinted or its analogue or derivative. The polymers may be used in a method for detection and/or assay of the imprinting molecule or metal ion.

### TECHNICAL FIELD

This invention relates to a molecularly imprinted polymer capable of binding organic molecules or metal ions and to applications using the polymer.

### BACKGROUND

Molecularly-imprinted polymers are polymers with an antibody-like ability to bind and discriminate between molecules. These are formed by the synthesis of cross-linked polymers in the presence of templates which may be the small molecule of interest and removal of the small molecule from the template to generate a structure complementary to the template structure or to an analogous structure. The polymer before removal of a small molecule may bind the small molecule covalently or it may be bound non-covalently.

To date commercialisation of such polymers has generally not been successful. One reason for this is that such polymers do not bind the target molecules with sufficient specificity in aqueous biological samples.

It is an object of this invention to provide a new binding material for use in detection of organic molecules or metal ions which can be used with aqueous samples, and/or biosensors comprising the new binding material and/or methods using these binding materials, or at least to provide the public with a useful choice.

### DISCLOSURE OF THE INVENTION

In one aspect, the invention provides an imprinted polymer imprinted with an organic molecule or a metal ion, wherein the matrix of said polymer has been prepared from one or more monomers including bilirubin or an analogue thereof.

In a further aspect the invention provides a method for preparing such an imprinted polymer comprising polymerising one or more monomers including bilirubin or an analogue or derivative thereof in the presence of the molecule or metal ion to be imprinted or an analogue or derivative thereof, and subsequently at least partly removing the molecule or ion to be imprinted or its analogue or derivative.

The imprinted polymers according to the invention can be prepared in a variety of ways. The common feature is that the imprinting molecule or ion is incorporated during the polymerisation or crosslinking process and then later removed. In one alternative bilirubin-containing polymers are crosslinked in the presence of the molecule or ion.

Preferably the polymerisation is an alkene polymerisation. Preferably in addition to bilirubin the mixture contains one or more further alkenes having more than one alkene group, for example monomers containing two acrylate or two methacrylate groups or one of each type of group or three or more groups independently selected from acrylate and methacrylate. These type of monomers serve as crosslinkers. The polymerisation may also include monoalkenes eg. methacrylic acid, vinylpyridines, hydroxyethylmethacrylate, acrylamide. These serve as comonomers.

Non-covalent interactions between the imprinting molecule and the polymer are generally used. The polymer is formed by adding the imprinting molecule during formation or crosslinking of the polymer. The polymer is selected so there will be electrostatic interaction, hydrogen bond formation or hydrophobic interactions with the imprinting molecule creating binding sites for the imprinting molecule.

Preferred noncovalently imprinted polymers include bilirubin-containing crosslinked polyacrylates and polymethacrylates, preferably bilirubin-containing crosslinked polymethacrylates. The preferred crosslinker is ethylenedimethacrylate. Preferably the mole ratio of comonomer to crosslinker is in the ratio 0:1 to 1:15 preferably 0:1 to 1:10. The preferred mole ratio of bilirubin to the crosslinker is 1:20 to 1:1, preferably 1:20 to 1:4.

In preferred embodiments of the invention the polymer to be used in the assay is ground repeatedly to reduce non-specific binding. Preferably the particle size of at least 50% by weight of the polymer is in the range 38 to 150 microns. More preferably more than 80% of the material consists of particles in that size range.

The above described polymers may be used in assays in which binding of the imprinting molecule is detected. These may be analogous to radioimmunoassays. For example radiolabelled imprinting molecule (for example [C¹⁴ or ³H] imprinting molecule) may be incorporated into a sample. Binding of the radioactive imprinting molecule to the polymer will be inversely related to the amount of imprinting molecule present in the sample. The binding of the imprinting molecule may be determined after separating the polymer from the liquid medium. This may conveniently be achieved by centrifugation.

Alternatively imprinting molecule binding to bilirubin-containing polymers may be detected by for example change in fluorescence of the polymer.

Another method for analysing imprinting molecules involves incorporation of the polymer into a biosensor. A preferred biosensor comprises an amperometric probe with an electrode, preferably molecularly imprinted polymer (M) coated platinum mesh. A reference probe is incorporated according to standard design techniques. Reference electrode materials include silver, gold, platinum or stainless steel. Preferred electrodes are Ag, Ag/AgCl combination. The electrodes may be connected to external points.

The probe assembly may be fitted within a body or housing to form an indicator probe. Such probes are exemplified in Example 2.

In a preferred embodiment of the invention, the imprinted polymer is formed by placing the polymerisation mixture on a surface, for example glass, a metallic surface or a membrane made from for example PTFE, mixed cellulose esters, polycarbonate, glass fibre or polypropylene with a 0.5 micron cutoff and allowed to polymerise. The resultant membrane can be used in biosensors.

In another aspect of the invention the concentration of imprinting molecule in biological samples is measured using an assay based on binding of the molecule onto a polymer previously imprinted with the molecule, either by optical or electrochemical detection.

Bilirubin binds small molecules, metals and proteins. Bilirubin can associate to a range of molecules due to its range of functional groups, and due to the fact it can wrap around other molecules. Typically the imprinting molecules (or molecules to be detected and/or assayed) are organic molecules generally with at least one hydrophilic group and having a molecular weight below 70,000 preferably below 10,000 more preferably below 3,000 and include proteins, peptides, steroid hormones and phenols. In addition, metal ions may also be measured using polymers of the invention, for example ferrous and ferric ions. Among the metal ions that may be assayed are arsenic and gold ions. In a preferred method the ions are cupric ions.

According to a further aspect of the present invention there is provided a method for the detection and/or assay of a compound comprising
a) contacting the sample to be tested with a polymer of the invention imprinted with the molecule or an analogue thereof,
b) measuring binding of the molecule to the polymer.

The invention also provides a corresponding method for the detection and/or assay of metal ions.

Certain preferred aspects of the invention will now be described in relation to the following non-limiting examples.

### BRIEF DESCRIPTIONS OF THE DRAWINGS

FIG. 1 shows percentage binding of rhodamine B to imprinted polymer plotted against amount (mg) of polymethacrylate polymer (classic imprinted polymer)where the solvent is (a) 40% methanol-water 0.5% acetic acid (b) acetonitrile and (c) dichloromethane. The symbols used are diamonds indicating the imprinted polymer and squares indicating the control polymer.

FIG. 2 shows binding to the polymer with and without bilirubin when the analyte is rhodamine B, rhodamine 6G and sulforhodamine B (bound/total). MAA polymer is a methacrylate polymer (shown as unshaded bars)—BRB is a bilirubin-containing polymer (shown as dark shaded bars). The left of each pair of bars shows the binding to imprinted polymer. The right hand side of each pair of bars shows binding to the non-imprinted polymer.

FIG. 3 shows rhodamine B binding (bound/total) to imprinted (diamond symbols) and non-imprinted (square symbols) bilirubin polymer in different methanol-water mixtures.

FIG. 4 shows cortisol binding to imprinted and non-imprinted polymers prepared with varying proportions of bilirubin and methacrylic acid. The data for imprinted and non-imprinted polymers is shown as unshaded and dark shaded bars respectively.

FIG. 5 shows a schematic representation of a probe of the current present invention.

FIG. 6 shows cortisol binding to non-imprinted bilirubin-containing polymer (CB) and cortisol imprinted bilirubin-containing polymer (CP). The binding (bound/total) is plotted against time (minutes). The unshaded bars and dark shaded bars show data for the imprinted and non-imprinted polymers respectively. The solvent is (a) water (b) 10% methanol and (c) 20% methanol.

FIG. 7 shows binding (bound/total) of copper ions to bilirubin-containing polymer and non-imprinted polymer (shown as unshaded and dark-shade bars respectively) at 1 hour and 4 hours.

### EXAMPLE 1

### Preparation of Polymers

The bilirubin-containing polymers were prepared using 0.05 mmoles template (rhodamine, cortisol, propofol); 0.2 mmoles bilirubin; 2 mmoles ethylenedimethylacrylic acid (EDMA); 1.5 mL dichloromethane (porogen); 20 mg 2,2′-azobisisobutyronitrile (AIBN) (initiator). All were put in a vial, dissolved, and thermally polymerised for 20 hours (70 degrees). For cortisol imprinting only, 0.2 mmoles of diisopropylethylamine was also included. The block of polymer was ground and sieved. The 38-150 micrometer fraction was kept and used in subsequent tests. The template was then removed using a Soxhlet extraction with a suitable solvent:
cortisol: methanol, 24 hours
rhodamine B and 6G: ethanol, 24 hours
sulforhodamine: ethanol, 48 hours
propofol: methanol, 24 hours

For the polymers having DMSO added as well, the Soxhlet time was increased by an extra 6 hours—in the same solvents.

### Assay

Tests: suspensions of polymers (amounts specified for each polymer) were made in the solvent chosen for testing (as specified in each case). 0.9 mL suspension came in contact with 0.1 mL of 0.5 mM tests solution in the same solvent. The solutions were allowed to reach equilibrium (20 hours on the shaker). 2 min centrifugation at 14,000 rpm was applied to settle the powders and the supernatant was tested by either HPLC (high performance liquid chromatography) or spectrophotometrically, depending on the template we were trying to test for.

Cortisol in all solvents was tested by HPLC, rhodamines by spectrophotometry and propofol by HPLC and spectrophotometry.

For all templates, the control classic non-covalent polymer was made at the same time and tested against the same conditions as the bilirubin one.

Control Classic polymers were prepared exactly the same as the bilirubin-containing polymers, but replacing the 0.2 mmoles bilirubin with 0.8 mmoles methacrylic acid (MAA)

Herein references are made to the MAA ones as 100% MAA and the bilirubin ones as 0% MAA Rhodamine polymers. The classic one was developed first, and the optimum binding conditions were developed on this polymer. Solvents tested for rhodamine polymers: acetonitrile, dichloromethane, and 40% methanol-0.5% acetic acid

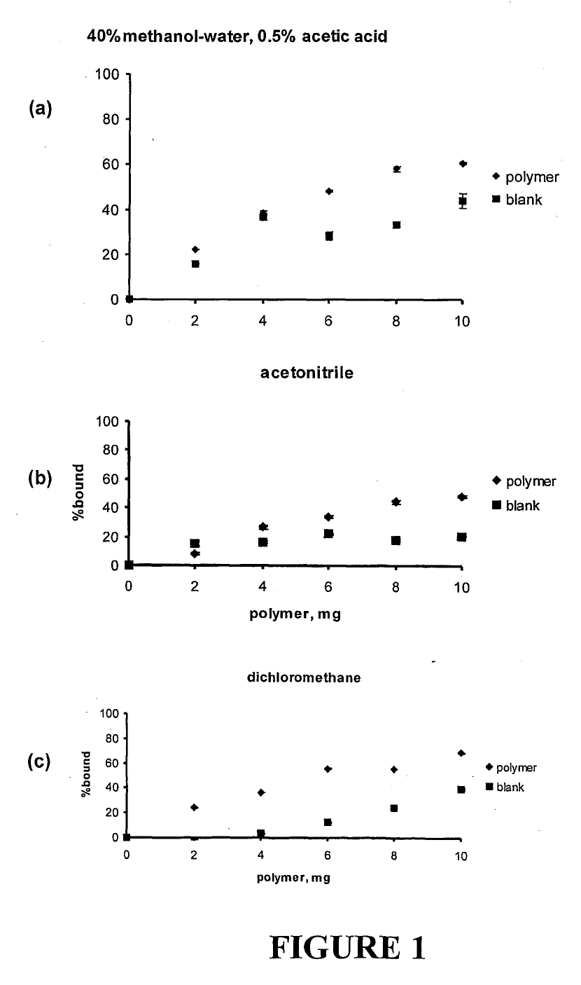

The results for the classic imprinted polymer with rhodamine B are shown in FIG. 1 for (a) 40% methanol-water 0.5% acetic acid (b) acetonitrile (c) dichloromethane. In each case a greater percentage of rhodamine B binds to the imprinted polymer than to the control polymer.

### Similar Results for Different Templates

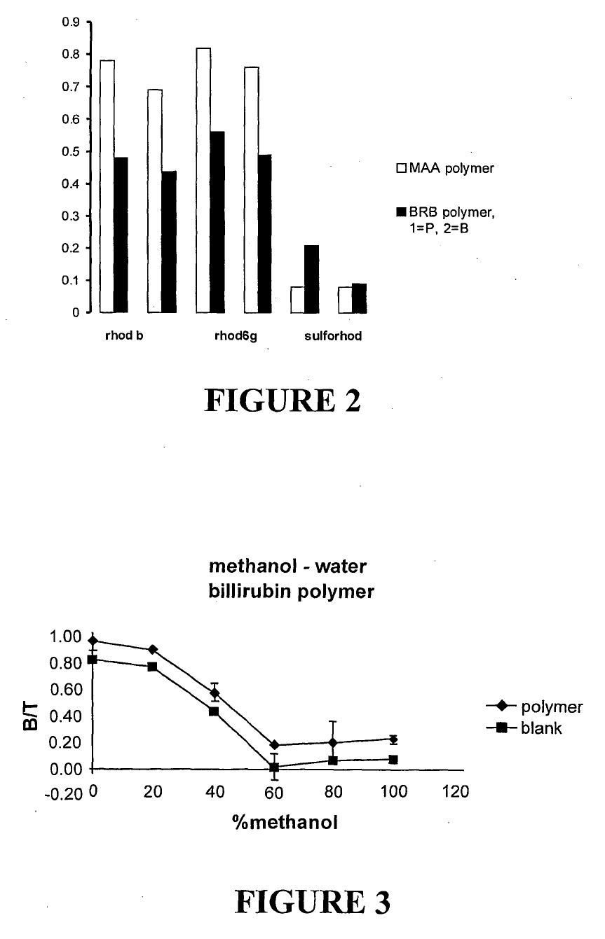

Observation: the bilirubin polymer binds better from aqueous solutions, and it changes fluorescence upon binding the template. FIG. 2 shows the binding of Rhodamine B, rhodamine 6G and sulforhodamine B to MAA polymer and bilirubin-containing polymer each both with and without molecular imprinting with rhodamine B. The solvent was 40% methanol-water. The specific binding of sulforhodamine B to the bilirubin-containing polymers was particularly high relative to the non-specific binding.

### Variation of Solvent

FIG. 3 shows the binding of rhodamine B to a rhodamine B imprinted bilirubin-containing polymer in solvents with different proportions of methanol and water. The binding was higher in all the mixtures for the imprinted polymer than for the corresponding polymer without rhodamine B imprinting.

### Cortisol Binding-Effect of Variation of Bilirubin Content

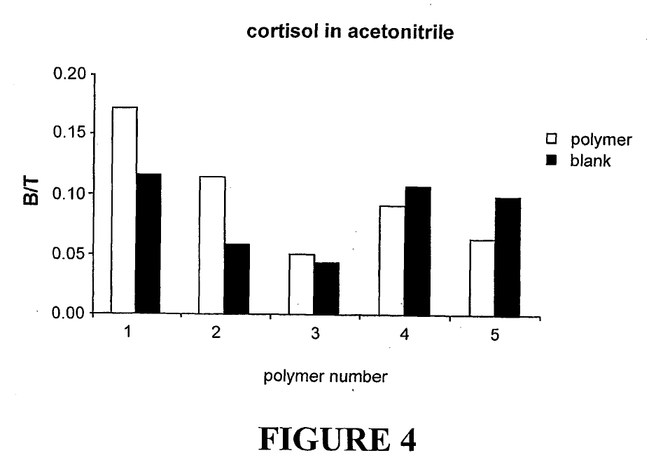

FIG. 4 shows the bound/total ratio for cortisol binding to polymers with the different proportions of bilirubin shown in Table 1. Specific binding of cortisol was higher in cortisol imprinted polymers than in non-imprinted controls when the bilirubin content was higher than the methacrylic acid content.

TABLE 1
Composition of the cortisol-imprinted polymers for FIG. 4

| Polymer Number | Bilirubin | Methacrylic acid | Diisopropylethylamine |
|---|---|---|---|
| 1 | 10 | 3 | 0 |
| 2 | 10 | 3 | 200 |
| 3 | 10 | 0 | 0 |
| 4 | 10 | 30 | 0 |
| 5 | 0 | 80 | 0 |

### EXAMPLE 2

The polymerisation procedure may be carried out as in Example 1. Then a known amount of liquid polymerisation mixture is placed on a PTFE membrane (Millipore, Fluoropore FHUP04700), 0.5 microns cutoff and allowed to polymerise (thermic or UV).

These sorts of membranes can be used in biosensors as a one-off “dip in” analysis that would give rapid and accurate results.

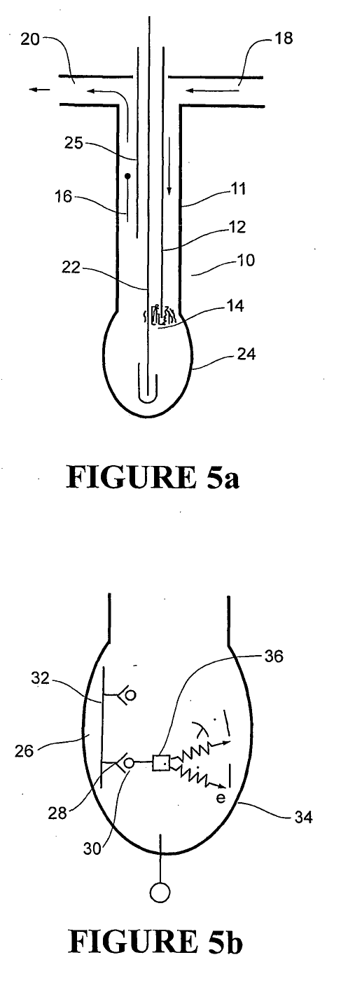

FIG. 5 offers a schematic representation of the probe components as detailed in the present invention. These include an inlet tube (18) that allows introduction of analyte into the probe which can be monitored in numerous forms, including but not exclusively by flow rates by on-line monitoring, a central body (11) of the probe (10) is included, constructed of known materials such as steels, alloys, plastics, glass in a concentric manner and including a selective membrane design (24) that separates the analysis actions within the probe (10) from the sample and/or substrate. Within the central body (11) of the probe (10) lies the sensor components (12, 16, 25) surrounded by, or in contact with, or directed towards analyte imprinted polymer (14).

The internal probe is separated by divider (22) into two chambers until a short distance prior to the actual separation membrane. The probe also consists of an outlet (20) with monitoring opportunities as described for the inlet This outlet also offers the opportunity for actual sample collection should it be desirable. The sensor arrangement within the probe (12,16,25) can be connected to amplifying, displaying and quantify devices including the provision for logging of data or radio-electric transmission to a receiver some distance away.

One probe of the invention depicted in FIGS. 5a and 5b comprises a response portion (26) comprising an area of receptors. These comprise imprinted polymer of the invention specific to the imprinted molecule (30), bound to a supporting substrate (32). The components are housed in a body (11) allowing fluid from the sample to access the response portion (26). The response portion (26) may be housed in the head of the body (11), while the bulk of equipment associated with evaluating the labeled standard can be positioned other than in the head to reduce its size.

The receptor may comprise imprinted polymer arranged around the base area of the probe in a number of formats. These may include formation of the polymer on the measuring electrode (12), which may be platinum mesh, gold, stainless steel, carbon, alloys or optic fibres coated with imprinted polymer, as a very thin layer or even a monolayer. Other methods of attaching the polymer are not excluded.

A fibre optic (25) delivers exciting electromagnetic radiation from a light source and also delivers emitted fluoresced light from the label of introduced standard at the surface of the response portion (26) to suitable electronic circuitry.

In FIG. 5b it can be seen that in use a molecule of interest (30) in the sample may selectively travel across a membrane (34) into the measurement part of the probe. Once there (30) may bind to an polymer of the invention (28) fixed within the probe. An introduced ligand (36) competitively binds to the same set of receptors (28). This introduced ligand (36) is then activated to produce energy proportional to the number of ligands (36) bound This energy is monitored, and measured to give a relative measure of (36) bound and therefore (30) bound. This relative measure is calibrated from the performance of the probe using standards of (36) and (30) in an in vitro calibration or in vivo internal standard test.

According to one method of use, the probe will be calibrated, typically in a sample of pure labeled standard to obtain a 100% reading. Known standards comprising known mixture of both labeled and non-labeled competitively binding substances may be used for calibration, or to obtain various data points for subsequent comparison and analysis. Calibration will normally occur in vitro, before and after use although in vivo calibration using internal standards is also possible. The probe, after will be placed in the sample and allowed to equilibrate. A standard of labeled substance is introduced to the sample or system being monitored, allowed to distribute and competitively bind at the receptor sites. After equilibration, meaningful data from the sensor portion may be collected and analysed.

### EXAMPLE 3

### Synthesis of a Cortisol-Imprinted Polymer

Preparation of polymer was as in example 1 but with no amine included in the composition of the polymer though. The test were performed the same as in Example 1, after the polymer was cleaned as for general procedure described in Example 1.

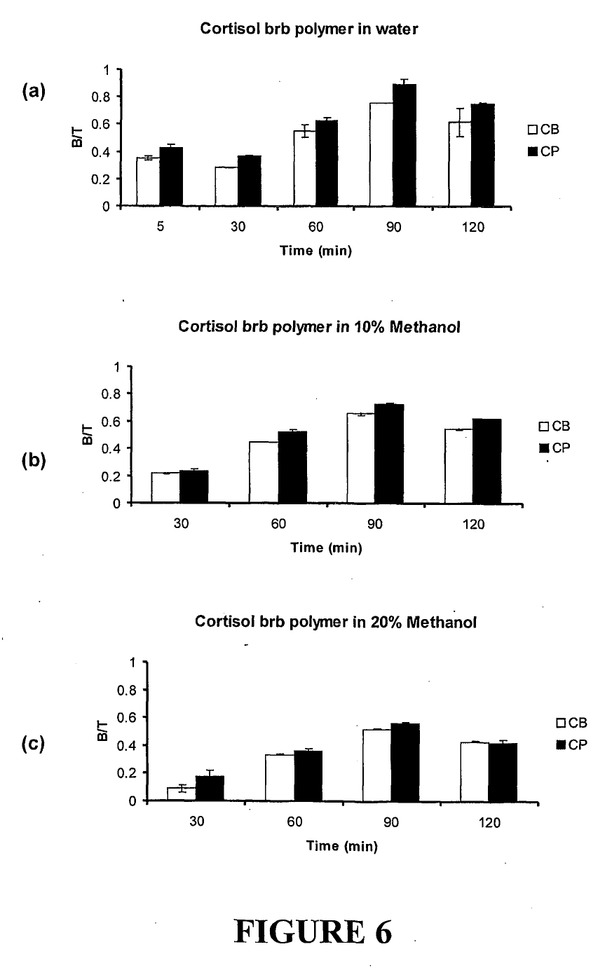

The polymers containing bilirubin are targeted to perform better in aqueous environments, so the tests were performed so as to test for recognition in water and high concentration aqueous environments that would mimic biological fluids. Cortisol-imprinted bilirubin polymers were equilibrated in water and solutions of 10% and 20% methanol in water and tested against a test solution developed in the same solvent. Results are shown below in FIG. 6. The imprinted polymers bound more cortisol than the non-imprinted Water was the solvent in which this effect was largest.

The classic imprinted polymers do not have recognition abilities in water as all binding is done through non-specific adsorption on the polymer and not through specific recognition. In the bilirubin-cortisol imprinted polymer, the binding occurring in water is performed in the active cavities. The same thing can be said about 10% and 20% methanol solutions in water. By comparison, in the classic imprinted polymers using methacrylic acid, binding starts occurring when methanol concentration in water exceeds 40%.

### EXAMPLE 4

### Chloramphenicol Imprinting

Polymers were prepared as described in Example 1 using chloramphenicol as the imprinting molecule (template). Assays were conducted as in Example 1. Chloramphenicol was assayed by spectrophotometry at 274 nm.

Chloramphenicol binding was higher for imprinted polymer relative to non-imprinted polymer when the solvent was water or up to 30% methanol. (See Table 2).

TABLE 2
Binding (Bound/Total) of Chloramphenicol to non-imprinted (Blank)
and Imprinted (Test) polymer in Aqueous Methanol solutions

| % Methanol | Blank | SD | Imprinted | SD |
|---|---|---|---|---|
| 0 | 0.392 | 0.005 | 0.634 | 0.032 |
| 10 | 0.312 | 0.007 | 0.486 | 0.007 |
| 20 | 0.232 | 0.003 | 0.345 | 0.014 |
| 30 | 0.220 | 0.007 | 0.309 | 0.033 |
| 40 | 0.151 | 0.003 | 0.123 | 0.008 |
| 50 | 0.101 | 0.004 | 0.061 | 0.003 |
| 60 | 0.100 | 0.030 | 0.013 | 0.014 |
| 70 | 0.178 | 0.019 | 0.090 | 0.006 |
| 80 | 0.090 | 0.042 | −0.024 | 0.038 |
| 90 | 0.090 | 0.022 | −0.040 | 0.022 |
| 100 | 0.141 | 0.010 | 0.024 | 0.010 |

### EXAMPLE 5

### Heavy Metal Imprinting

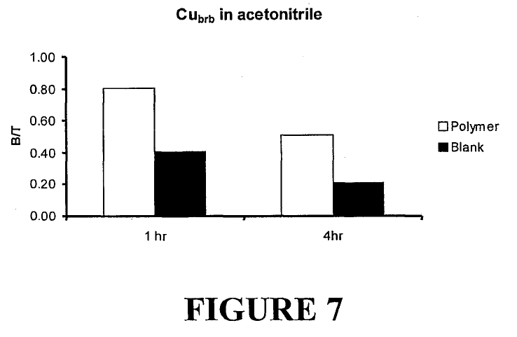

Copper (CuII) was used as a model ion for heavy metal imprinting. Copper was trialed as part of different salts (sulfate, chloride) and imprinting was performed with bilirubin directly, as the ‘classic’ system would be too complicated to perform, involving complex coordination sites in the active cavities. Copper chloride was placed in contact with bilirubin and crosslinkers and polymerised as per example 1, then extracted by strongly varying the pH of the solution (rinses with 2M HCl and 1M sodium carbonate). Polymers were tested in acetonitrile solutions and aqueous solutions, against chlorides and sulfates of copper (II) salts. Results for the currently preferred solvent, acetonitrile are shown in FIG. 7. Binding to the imprinted polymers was approximately double that when the non-imprinted polymer was used.

### EXAMPLE 6

### Synthesis of a Protein-Imprinted Polymer

Polymers were prepared using 2 ml acrylamide solution, containing 50% acrylamide and 1.3% bisacrylamide (w/v), 10 mg bilirubin, 50 microliters protein (bovine serum albumin) in water (1 mg/ml solution), 10 mg ammonium persulfate and 10 microliters TEMED (N,N,N′,N′-tetramethylethylenediamine). The blanks were prepared in the same style, but with no protein. The polymer gels were formed as discs on the bottom of vials. The polymers were soaked in the vials with 2M HCl for 2 hours and then rinsed with 0.5M NaHCO₃ to remove protein. The discs were then generally kept in water. If they dried out at least 48 hours was allowed for re-equilibration with water before any tests were carried out.

Assays for binding of the protein to the polymers were carried out as in Example 1 except that discs were used. Absorbance at 280 nm was used to detect the protein. Substantial binding of the protein was found when the imprinted-polymer containing bilirubin, relative to when the corresponding non-imprinted polymer was used as indicated by lower levels of protein in the supernatant (Table 3).

TABLE 3
Supernatant Protein after contact with polymer

| Polymer | Run 1 | Run 2 |
|---|---|---|
| Non-imprinted bilirubin polymer | 0.698 | 0.71 |
| Protein-imprinted bilirubin polymer | 0.464 | 0.457 |

The term “comprising” as used in this specification means ‘consisting at least in part of’, that is to say when interpreting statements in this specification which include that term, the features, prefaced by that term in each statement, all need to be present but other features can also be present

It should be noted that the invention can be carried out with numerous modifications and variations and that the above Examples are by way of illustration only. For example the invention may be carried out using other molecules or ions and the polymers used may be prepared using different monomers and/or proportions and/or crosslinkers.

1-29. (canceled)

30. An imprinted polymer imprinted with an organic molecule or a metal ion, wherein the matrix of the polymer has been prepared from one or more monomers including bilirubin or an analogue thereof.

31. The imprinted polymer as claimed in claim 30 wherein the imprinting is with a molecule with at least one hydrophilic group and a molecular weight below 70,000.

32. The imprinted polymer as claimed in claim 30 wherein the polymer is imprinted with a molecule selected from the group comprising a protein, a peptide, a steroid hormone and a phenol.

33. The imprinted polymer as claimed in claim 30 wherein the imprinting is with a metal ion.

34. The imprinted polymer of claim 30 that is the product of an alkene polymerisation.

35. The imprinted polymer as claimed in claim 34 wherein the polymer is the product of alkene polymerisation of a mixture comprising bilirubin and at least one further compound with more than one alkene group.

36. The imprinted polymer as claimed in claim 35 wherein the at least one further alkene includes a monomer including at least two groups independently selected from acrylate and methacrylate.

37. The imprinted polymer as claimed in claim 30 wherein the imprinting molecule was non-covalently bound during the formation or cross-linking of the polymer and subsequently at least partially removed by extraction with a solvent.

38. The imprinted polymer as claimed in claim 30 wherein the polymer is cross-linked using ethylene dimethacrylate.

39. The imprinted polymer as claimed in claim 30 wherein the mole ratio of comonomer to crosslinker is in the ratio 0:1 to 1:15.

40. The imprinted polymer as claimed in claim 30 wherein the polymer was prepared using a crosslinker and the mole ratio of bilirubin to the crosslinker is 1:20 to 1:1.

41. The imprinted polymer as claimed in claim 30 wherein the polymer is in form of particles, the particle size of at least 50% by weight of the polymer being in the range 38-150 microns.

42. The method for preparing an imprinted polymer as claimed in claim 30 comprising polymerising one or more monomers including bilirubin or an analogue or derivative thereof in the presence of the molecule or metal ion to be imprinted or an analogue or derivative thereof, and subsequently at least partly removing the molecule or metal ion to be imprinted or its analogue or derivative.

43. The method as claimed in claim 42 wherein the imprinting molecule is a molecule with at least one hydrophilic group and a molecular weight below 70,000.

44. The method as claimed in claim 43 wherein the polymer is imprinted with a molecule selected from the group comprising a protein, a peptide, a steroid hormone and a phenol.

45. The method as claimed in claim 42 wherein the imprinting is with an ion

46. The method as claimed in claim 42 wherein polymerisation is alkene polymerisation.

47. The method as claimed in claim 46 wherein a mixture comprising bilirubin and at least one further compound with more than one alkene group is polymerised.

48. The method as claimed in claim 47 wherein said at least one further alkene includes a monomer including at least two groups independently selected from acrylate and methacrylate.

49. The method as claimed in claim 42 wherein the imprinting molecule is non-covalently bound during the formation or cross-linking of the polymer and is subsequently at least partially removed by extraction with a solvent.

50. The method as claimed in claim 42 wherein the polymer is cross-linked using ethylene dimethacrylate.

51. The method as claimed in claim 42 wherein the mole ratio of comonomer to crosslinker is in the ratio 0:1 to 1:15.

52. The method as claimed in claim 42 wherein mole ratio of bilirubin to the crosslinker is 1:20 to 1:1.

53. The method as claimed in claim 42 wherein the imprinted polymer is ground to a particle size of at least 50% of a particle by weight of the polymer is in the range 38-150 microns.

54. A method for the detection and/or assay of a compound or a metal ion comprising
a. contacting the sample to be tested with an imprinted polymer, imprinted with a metal ion or an organic molecule, the matrix of the polymer having been prepared from one or more monomers including bilirubin or an analogue or derivative thereof,
b. measuring binding of the molecule to the polymer.

55. The method as claimed in claim 54 wherein the substance to be detected and/or assayed is a compound selected from the group comprising a protein, a peptide, a steroid hormone and a phenol.

56. The method as claimed in claim 54 wherein the substance to be detected is a metal ion.

57. The method as claimed in claim 55 wherein the compound is selected from, cortisol, propofol and chloramphenicol.

58. The method as claimed in claim 56 wherein the ion is a cupric ion.
